(19) United States
(12) Patent Application Publication
(10) Pub. No.: US 2023/0044621 A1
(43) Pub. Date: Feb. 9, 2023

(54) APPARATUS AND METHOD FOR GENE AMPLIFICATION

(71) Applicants: SAMSUNG ELECTRONICS CO., LTD., Suwon-si (KR); AJOU UNIVERSITY INDUSTRY-ACADEMIC COOPERATION FOUNDATION, Suwon-si (KR)

(72) Inventors: Won Jong JUNG, Seoul (KR); Kwon-Young CHOI, Suwon-si (KR); Kak NAMKOONG, Seoul (KR); Hyuna PARK, Suwon-si (KR); Hyeong Seok JANG, Seoul (KR)

(73) Assignees: SAMSUNG ELECTRONICS CO., LTD., Suwon-si (KR); AJOU UNIVERSITY INDUSTRY-ACADEMIC COOPERATION FOUNDATION, Suwon-si (KR)

(21) Appl. No.: 17/535,204

(22) Filed: Nov. 24, 2021

(30) Foreign Application Priority Data
Aug. 5, 2021 (KR) 10-2021-0103136
Aug. 26, 2021 (KR) 10-2021-0112883

(51) Int. Cl. B01L 3/00; C12Q 1/686; B01L 7/00

(52) U.S. Cl. CPC B01L 3/508; C12Q 1/686; B01L 7/52; B01L 2200/025; B01L 2200/0647; B01L 2200/10; B01L 2200/16; B01L 2300/0663; B01L 2300/0893; B01L 2300/168; B01L 2300/1861; B01L 2300/1822; G01N 21/47

(57) ABSTRACT

An apparatus for gene amplification includes a gene amplification chip including a well configured to accept a sample that is loaded into the well; the gene amplification chip being configured to: thermally dissolve the sample in the well so that a microbe present in the sample is thermally dissolved in the well to release genes in the microbe; and amplify the released genes in the well. The apparatus for gene amplification also includes a temperature controller configured to control a thermal dissolution temperature and a gene amplification temperature of the well.

### CROSS-REFERENCE TO RELATED APPLICATION(S)

This application is based on and claims priority to Korean Patent Application No. 10-2021-0103136, filed on Aug. 5, 2021 in the Korean Intellectual Property Office, and Korean Patent Application No. 10-2021-0112883, filed on Aug. 26, 2021 in the Korean Intellectual Property Office, the disclosures of which are incorporated by reference herein in their entirety.

### BACKGROUND

### 1. Field

The following description relates to gene amplification, and in particular to an apparatus and method for gene amplification.

### 2. Description of the Related Art

Clinical or environmental samples may be analyzed by a series of biochemical, chemical, and mechanical treatment processes. Development of technology for diagnosis or monitoring of biological samples, in which molecular diagnosis based on nucleic acid has excellent accuracy and sensitivity, is increasingly used in various applications, such as diagnosis of infectious diseases or cancer, pharmacogenomics, development of new drugs, and the like. Microfluidic devices are widely used in order to analyze samples conveniently and accurately according to various purposes.

### SUMMARY

According to an aspect of the disclosure, an apparatus for gene amplification may include a gene amplification chip comprising a substrate including a well the well configured to accept a sample that is loaded into the well; the gene amplification chip being configured to: thermally dissolve a microbe present in the sample, to release genes in the microbe, by heating the sample in the well to a thermal dissolution temperature; and amplify the released genes by heating the sample in the well to a gene amplification temperature; and a temperature controller configured to control a temperature of the well to be the thermal dissolution temperature and the gene amplification temperature.

A diameter of the well may be larger than or equal to a diameter of the microbe, and may be less than or equal to 10000 times the diameter of the microbe.

Each of the diameter and a depth of the well may be in a range of 1 nm to 1000 μm.

A volume of the well may be 1 nL or less.

The apparatus may further include an optical device configured to, while the gene amplification is being performed in the well or after the gene amplification is complete, measure an optical signal that is scattered or reflected from the sample in the well; and a processor configured to detect the amplified genes by analyzing the measured optical signal.

The apparatus may further include a time controller configured to control a time of the thermal dissolution to be 10 minutes or less, and a time of the gene amplification to be 120 minutes or less.

The gene amplification chip may be further configured to, in response to the microbe being RNA virus, reverse transcribe RNA, that is released after the RNA virus is thermally dissolved in the well, using a reverse transcriptase in the wells.

The temperature controller may be further configured to control the thermal dissolution temperature of the well based on a predetermined activation temperature range of the used reverse transcriptase; and the apparatus for gene amplification further comprises a time controller configured to control a time of the reverse transcription to be 20 minutes or less.

A viral membrane softening agent may be used for the thermal dissolution.

The viral membrane softening agent may include at least one of ethanol, isopropyl alcohol, methanol, 1-propanol, 2-propanol, 1-butanol, 2-butanol, triethylamine, dimethylformamide, hexamethylphosphoric triamide, dimethyl sulfoxide, acetone, acetonitrile, pyridine, metal particle, detergent, ethyl acetate, or hexanol.

The gene amplification chip further comprising a photothermal film and the apparatus further comprising a heat source comprising at least one of: an optical heating element configured to emit light, the photothermal film being configured to heat the sample in the well by generating heat from the light emitted by the optical heating element; or an electrical heating element comprising a Peltier element, the electric heating element being configured to heat the sample in the well. The temperature controller controls the thermal dissolution temperature and the gene amplification temperature by using the heat source.

The gene amplification may include at least one of polymerase chain reaction (PCR) amplification and isothermal amplification.

The well may include a plurality of wells in the range of one to a hundred thousand wells.

The gene amplification chip may include a substrate on which the well is formed. The well may have a through hole shape and passes through the substrate in a direction from an upper surface of the substrate to a lower surface of the substrate.

According to another aspect of the disclosure, a method of gene amplification, the method may include loading a sample comprising a microbe into a well of a substrate; thermally dissolving the microbe in the loaded sample to release genes from the microbe by controlling a temperature of the well to be a thermal dissolution temperature; and amplifying the released genes in the well by controlling the temperature of the well to be a gene amplification temperature.

The method may further include emitting light onto the sample of the well while the gene amplification is performed in the well or after the gene amplification is complete; measuring an optical signal that is scattered or reflected from the sample in the well; and detecting the amplified genes by analyzing the measured optical signal.

The thermally dissolving of the microbe in the wells may include controlling a time of the thermal dissolution to be 10 minutes or less; and the amplifying of the released genes in the wells may include controlling a time of the gene amplification to be 120 minutes or less.

The method may further include, in response to the microbe being RNA virus, performing reverse transcription on RNA, released after the RNA virus is thermally dissolved in the well, using a reverse transcriptase in the well. The thermally dissolving of the microbe in the well may include controlling the thermal dissolution temperature of the well based on a predetermined activation temperature range of the used reverse transcriptase; and the reverse transcription may include controlling a time of the reverse transcription to be 20 minutes or less.

The thermally dissolving of the microbe in the well may include using a viral membrane softening agent.

The thermally dissolving of the microbe in the well and the amplifying of the released genes in the well may include controlling the temperature of the well to be the thermal dissolution temperature and the gene amplification temperature, respectively, by using a heat source comprising at least one of: an optical heating element configured to emit light on a photothermal film on the substrate, the photothermal film being configured to heat the sample in the well by generating heat from the light emitted from the optical heating element; or an electrical heating element including a Peltier element.

### BRIEF DESCRIPTION OF THE DRAWINGS

The above and other aspects and features of certain embodiments of the disclosure will be more apparent from the following description, taken in conjunction with the accompanying drawings, in which:

FIG. 1A is a block diagram of an apparatus for gene amplification according to an embodiment.

FIG. 1B is a diagram showing a correlation between a virus concentration and a number of amplified genes per volume according to an embodiment.

FIGS. 1C through 1E are diagrams showing examples in which a temperature controller controls temperature of wells according to various embodiments.

FIG. 2A is a block diagram showing an apparatus for gene amplification according to another embodiment.

FIG. 2B is a diagram showing a substrate and a plurality of wells formed in a through hole shape on the substrate according to an embodiment.

FIG. 2C is a diagram showing a cross-section of the substrate on which a photothermal film is disposed according to an embodiment.

FIG. 3 is a block diagram showing an apparatus for gene amplification according to yet another embodiment.

FIG. 4 is a perspective view of an apparatus for gene amplification according to an embodiment.

FIG. 5 is a flowchart of a method of gene amplification according to an embodiment.

FIG. 6 is a flowchart of a method of gene amplification according to another embodiment.

### DETAILED DESCRIPTION

Details of example embodiments are included in the following detailed description and drawings. Advantages and features of the present disclosure, and a method of achieving the same will be more clearly understood from the following example embodiments described in detail with reference to the accompanying drawings. Throughout the drawings and the detailed description, unless otherwise described, the same drawing reference numerals will be understood to refer to the same elements, features, and structures.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising,” “comprises,” “includes,” “has,” “having,” or “including” will be understood to imply the inclusion of stated elements but not the exclusion of any other elements. Also, the terms, such as ‘unit’ or ‘module’, etc., should be understood as a unit that performs at least one function or operation and that may be embodied as hardware, software, or a combination thereof. Throughout the disclosure, the expression “at least one of a, b or c” indicates only a, only b, only c, both a and b, both a and c, both b and c, all of a, b, and c, or variations thereof.

Hereinafter, various embodiments of a microfluidic chip, and an apparatus and method for detecting biomolecules will be described in detail with reference to the accompanying drawings.

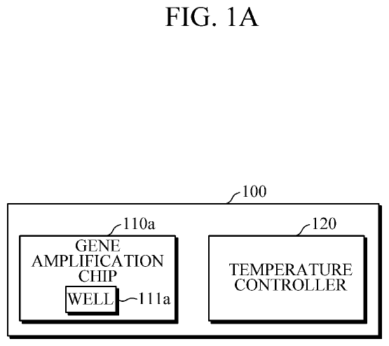

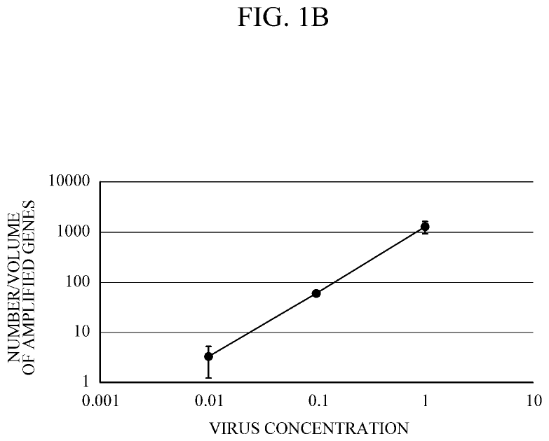

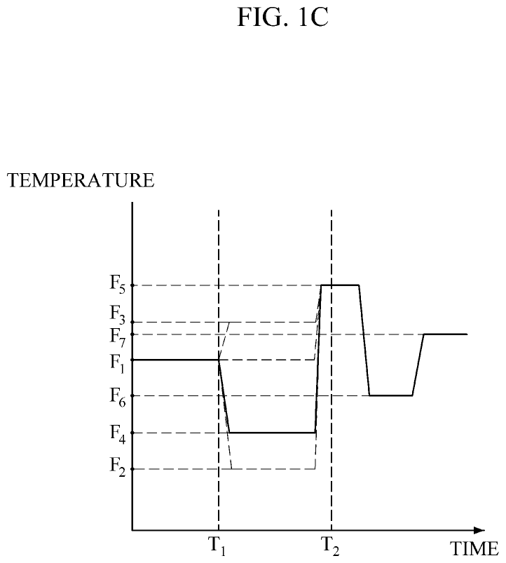

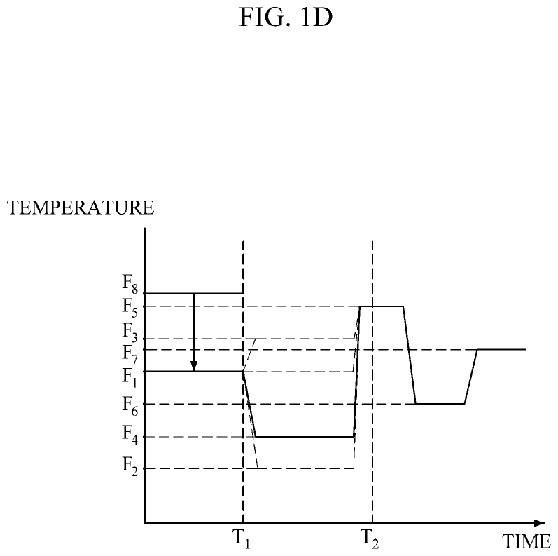

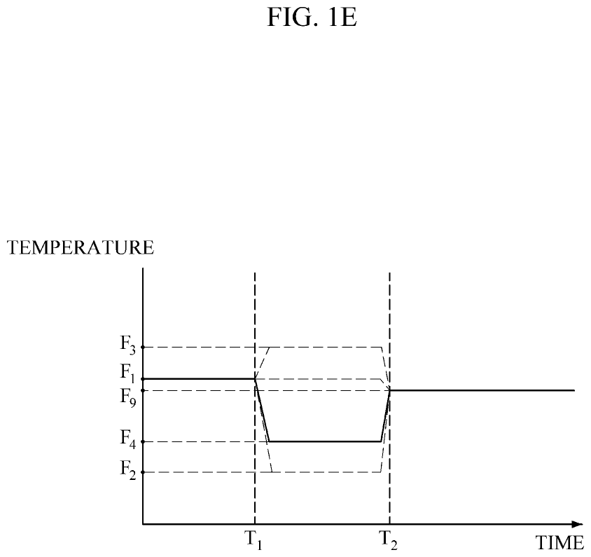

FIG. 1A is a block diagram of an apparatus for gene amplification according to an embodiment.

Referring to FIG. 1A, the apparatus 100 for gene amplification may include a gene amplification chip 110a and a temperature controller 120.

The gene amplification chip 110a may include a well 111a, into which a sample can be loaded.

The sample may be a specimen or a solution in which the specimen is diluted. For example, the sample may be bio-fluids, including at least one of respiratory secretions, blood, urine, perspiration, tears, saliva, etc., or a swab sample of the upper respiratory tract, or a solution of the bio-fluid or the swab sample dispersed in other medium. In this case, the other medium may include water, saline solution, alcohol, phosphate buffered saline solution, vital transport media, etc., but is not limited thereto.

The sample may include a duplex of one or more of ribonucleic acid (RNA) virus, deoxyribonucleic acid (DNA) virus, peptide nucleic acid (PNA) virus, and locked nucleic acid (LNA) virus, and microbes such as bacteria, pathogen, germ, oligopeptide, protein, toxin, etc., but the sample is not limited thereto. In this case, the microbes may contain genes. For example, the RNA virus may contain RNA within a membrane of the RNA virus, and the DNA virus may contain DNA within a membrane of the DNA virus.

A diameter of the well 111a may be larger than or equal to a diameter of a microbe, e.g., the RNA or DNA virus (to provide space for the microbe to be inserted), and may be less than or equal to 10000 times the diameter of the microbe (so the microbe can be readily detected), but is not limited thereto. For example, the diameter and depth of the well 111a may be in a range of from 1 nm to 1000 μm, but is not limited thereto, and may be changed to various values. A volume of the well 111a may be 1 nL or less, but is not limited thereto.

The numerical characteristics, such as the diameter, depth, volume, etc., of the well 111a may be changed to various values based on the size of pixels of a detector included in an optical device, and/or the sample size.

The gene amplification chip 110a may include a well array having a plurality of wells 111a. In this case, the number of wells 111a of the well array may be a hundred thousand or less, but is not limited thereto. As the gene amplification chip 110a may include the well array having the plurality of wells 111a, digital PCR may be performed, such that a sample contained in the respective wells 111a may be concentrated at a higher rate, and a purification effect for filtering impurities may be provided.

Concentrations and the purification effect, resulting from the gene amplification chip 110a including the well array, may lead to an increase in efficiency of gene amplification. The following Table 1 shows results of experiments, conducted three times in total, regarding the number/volume (μL) of the amplified genes according to a concentration of a loaded virus.

TABLE 1

| Virus | Number/volume (μL) of amplified genes | | |
|---|---|---|---|
| concentration | First time | Second time | Third time |
| 0.01 | 2.648 | 5.51 | 1.552 |
| 0.1 | 56.939 | 63.996 | 60.383 |
| 1 | 928.88 | 1298.2 | 1615.9 |

The experiment results of Table 1 may be expressed in a graph as illustrated in FIG. 1B. FIG. 1B is a diagram illustrating a correlation between the virus concentration and the number of amplified genes per volume, in which the horizontal axis indicates the concentration of the loaded virus, and the vertical axis indicates the number of amplified genes per volume (μL).

Referring to Table 1 and FIG. 1B, it can be seen that as the concentration of the loaded virus, e.g., RNA virus, increases, the number of the amplified genes per volume (μL) increases. Accordingly, the gene amplification chip 110a including the well array may provide the concentrations and purification effect, thereby increasing the efficiency of gene amplification. The temperature controller 120 may control temperature of the apparatus 100 for gene amplification. For example, the temperature controller 120 may control temperature of the sample, injected into a sample inlet of the apparatus 100 for gene amplification, to be maintained at an isothermal temperature of 95° C. or higher, or may control temperature of the wells 111a included in the gene amplification chip 110a to be within a predetermined range.

The temperature controller 120 may include a temperature sensor disposed inside or outside of the gene amplification chip 110a and measuring temperature of the wells 111a. In this case, the temperature sensor may include a thermocouple having a bimetal junction generating temperature-dependent EMF, a resistive thermometer including materials having electrical resistance proportional to temperature, thermistors, an IC temperature sensor, a quartz thermometer, and the like, but is not limited thereto.

A microbe present in the sample, which is loaded into the wells 111a, may be thermally dissolved in the wells 111a to release genes, and the released genes are reverse-transcribed and then amplified. In this case, according to elapsed time, the temperature controller 120 may control the temperature of the wells 111a to be each of a thermal dissolution temperature, a reverse transcription temperature, and a gene amplification temperature. FIGS. 1C and 1D show an embodiment in which the temperature controller 120 controls the temperature of the wells 111a to be each of the thermal dissolution temperature, the reverse transcription temperature, and the gene amplification temperature.

FIGS. 1C through 1E are diagrams showing examples in which the temperature controller 120 controls temperature of the wells 111a according to various embodiment.

In FIGS. 1C through 1E, the horizontal axis indicates a period from a time point 0 when a sample is loaded into the wells 111a to a time point when gene amplification ends. In this case, it is shown that the thermal dissolution occurs during a period from 0 to T₁, the reverse transcription occurs during a period from T₁ to T₂, and the gene amplification occurs during a period from T₂ to the end point.

Referring to FIG. 1C, the temperature controller 120 may control the temperature of the wells 111a to be the thermal dissolution temperature of F₁ during a predetermined period from 0 to T₁.

A viral membrane, or shell of a microbe present in the sample loaded into the wells 111a may be removed by the thermal dissolution. For example, if the microbe is the DNA virus, the viral membrane of the DNA virus may be removed by the thermal dissolution, such that DNA, i.e., genes inside the virus, may be released; and if the microbe is the RNA virus, the viral membrane of the DNA virus may be removed by the thermal dissolution, such that RNA, i.e., genes inside the virus, may be released.

The thermal dissolution temperature F₁, controlled by the temperature controller 120, may vary depending on the type of microbe contained in the loaded sample, an activation temperature range of a reverse transcriptase, or a user's manipulation of the apparatus for gene amplification. While FIG. 1C shows that the thermal dissolution temperature is an isothermal temperature F₁ during the predetermined period from 0 to T₁, but the thermal dissolution temperature is not limited thereto and may increase or decrease within a predetermined range during the predetermined period from 0 to T₁.

For example, the temperature controller 120 may control the thermal dissolution temperature based on the reverse transcription temperature. In this case, the temperature controller 120 may control the thermal dissolution temperature of the wells 111a based on a predetermined activation temperature range of a reverse transcriptase used to perform reverse transcription. Referring to FIG. 1C, if an activation temperature range of the reverse transcriptase used is from F₂ to F₃, the temperature controller 120 may control the thermal dissolution temperature to be within a temperature range of F₂ to F₃. In this case, while FIG. 1C shows that the thermal dissolution temperature is an isothermal temperature F₁ in a temperature range of from F₂ to F₃, but the thermal dissolution temperature is not limited thereto and may partially increase or decrease within the temperature range of from F₂ to F₃ during the predetermined period from 0 to T₁.

In this case, if the thermal dissolution temperature required for thermal dissolution of a microbe exceeds the predetermined activation temperature range of the reverse transcriptase, a viral membrane softening agent may be used. The process of controlling the thermal dissolution temperature by using the viral membrane softening agent will be described below with reference to FIG. 1D.

FIG. 1D is a diagram explaining a method of controlling the thermal dissolution temperature by using a viral membrane softening agent in the case where the thermal dissolution temperature required for thermal dissolution of a microbe exceeds the predetermined activation temperature range of the reverse transcriptase. Referring to FIG. 1D, if the thermal dissolution temperature required for thermal dissolution of a microbe exceeds the predetermined activation temperature range of F₂ to F₃ of the reverse transcriptase, e.g., in the case where temperature of F₈ is required, the thermal dissolution temperature required for the sample may be reduced to, e.g., F₁. By using the viral membrane softening agent, the temperature controller 120 may control the thermal dissolution temperature to be within a predetermined range, e.g., any temperature within the predetermined activation temperature range of F₂ to F₃ of the reverse transcriptase, thereby improving the efficiency of thermal dissolution.

The viral membrane softening agent may include ethanol, isopropyl alcohol, methanol, 1-propanol, 2-propanol, 1-butanol, 2-butanol, triethylamine, dimethylformamide, hexamethylphosphoric triamide, dimethyl sulfoxide, acetone, acetonitrile, pyridine, metal particle, and organic solvent such as detergent, ethyl acetate, hexanol, and the like, but is not limited thereto.

Referring back to FIG. 1C, the temperature controller 120 may control temperature of the wells 111a after the thermal dissolution to be the reverse transcription temperature during a predetermined period from T₁ to T₂. In this case, genes, e.g., RNA, released during the thermal dissolution, may be reverse transcribed.

A reverse transcriptase may be used for the reverse transcription, in which case the temperature controller 120 may control the reverse transcription temperature to be any temperature within the activation temperature range of F₂ to F₃ of the reverse transcriptase, e.g., to be F₄ as shown in FIG. 1C. In this case, the activation temperature range of the reverse transcriptase may be predetermined according to the reverse transcriptase used.

While FIG. 1C shows temperature during the reverse transcription is the isothermal temperature of F₄, the temperature is not limited thereto and may partially increase within a range of from F₂ to F₃ while the reverse transcription is performed.

The temperature controller 120 may control the temperature of the wells 111a after the thermal dissolution or the reverse transcription to be the gene amplification temperature during a predetermined period from T₂ to an end point. In this case, the thermally dissolved gene and/or reverse transcribed gene may be amplified within the wells 111a.

The gene amplification reaction may include at least one of polymerase chain reaction (PCR) amplification and isothermal amplification, but is not limited thereto.

In this case, according to the type of gene amplification reaction, the temperature controller 120 may vary the gene amplification temperature of the wells 111a. For example, if the gene amplification reaction is the PCR amplification, the temperature controller 120 may control the temperature of the wells 111a based on thermal cycling, and if the gene amplification reaction is the isothermal amplification, the temperature controller 120 may control the temperature of the wells 111a to be maintained at a specific temperature.

For example, FIGS. 1C and 1D are diagrams explaining an embodiment in which the temperature controller 120 controls temperature based on thermal cycling of the PCR amplification reaction during a predetermined period from T₂ to the end point. Referring to FIGS. 1C and 1D, the temperature controller 120 may control the temperature of the wells 111a for denaturation during the PCR amplification reaction to be F₅ for a predetermined period of time, may reduce the temperature to F₆ for annealing, and then may increase the temperature again to F₇ for extension. By performing the PCR amplification reaction based on thermal cycling, the genes in the sample may be amplified.

FIG. 1E shown another embodiment in which the temperature controller 120 controls temperature based on the isothermal amplification reaction during a predetermined period from T₂ to the end point. Referring to FIG. 1E, the temperature controller 120 may control the temperature of the wells 111a to be maintained at an isothermal temperature, e.g., F₉, during a predetermined period of time, in which case an error may occur within a predetermined range based on the temperature of F₉.

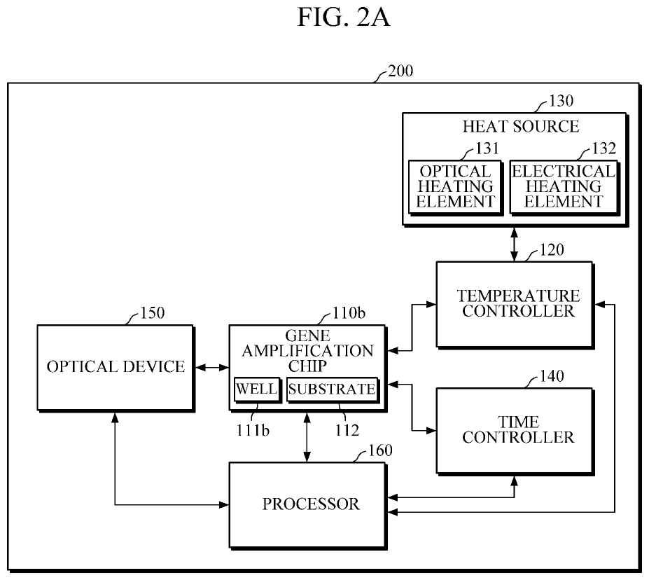

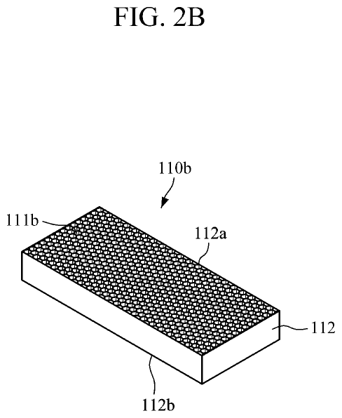

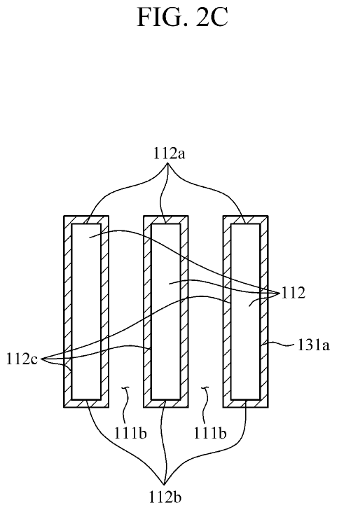

FIG. 2A is a block diagram showing an apparatus for gene amplification according to another embodiment.

Referring to FIG. 2A, the apparatus 200 for gene amplification may include a gene amplification chip 110b, a temperature controller 120, a heat source 130, a time controller 140, an optical device 150, and a processor 160.

Wells 111b of the gene amplification chip 110b may be formed on a substrate 112 included in the gene amplification chip 110b.

The substrate 112 may be made of any one of an inorganic matter, such as silicon (Si), glass, polymer, metal, ceramic, graphite, etc., and acrylic material, polyethylene terephthalate (PET), polycarbonate, polystylene, and polypropylene, but is not limited thereto.

The wells 11b may be formed in a through hole shape on the substrate 112. Referring to FIG. 2B, a structure of the wells 111b formed in a through hole shape on the substrate 112 will be described below.

FIG. 2B is a diagram showing the substrate 112 and the wells 111b formed in a through hole shape on the substrate 112 according to an embodiment. Referring to FIG. 2B, the gene amplification chip 110b includes an array of wells 111b formed in a through shape, and the substrate 112.

In this case, the substrate may have an upper surface 112a and a lower surface 112b, and the wells 111b may pass through the substrate in a direction from the upper surface 112a to the lower surface 112b, but is not limited thereto. In this case, the through hole may have a cylindrical or hexagonal prism shape, but its shape is not limited thereto, and may be formed in various shapes such as other polygonal prism and the like. In the case where the gene amplification chip 110b includes an array of wells 111b, the array of wells 111b having the through hole shape may have at least 20 to 100 thousand wells 111b, but the number of the wells 111b is not limited thereto.

Referring back to FIG. 2A, when controlling the temperature of the wells 111b, the temperature controller 120 may use the heat source 130 disposed inside or outside of the apparatus 200 for gene amplification. In this case, the heat source 130 may include at least one of an optical heating element 131 and an electrical heating element 132, but is not limited thereto.

The optical heating element 131 may include a material for generating heat by using received light, and an example thereof may include a photothermal film. In this case, the photothermal film may be disposed on the substrate 112 of the gene amplification chip 110b. An arrangement of the photothermal film disposed on the substrate 112 will be described below with reference to FIG. 2C.

FIG. 2C is a diagram showing a side surface of a substrate on which a photothermal film is disposed according to an embodiment.

Referring to FIG. 2C, the gene amplification chip may include a substrate 112, a substrate upper surface 112a, a substrate lower surface 112b, a well 111b having a through hole shape, a partition wall 112c of the well 111b defining the through hole shape, and a photothermal film 131a.

FIG. 2C illustrates an example in which the photothermal film 131a is disposed on the substrate upper surface 112a, the substrate lower surface 112b, and the partition wall 112c of the well 111b having the through hole shape. However, the arrangement is not limited thereto, and the photothermal film 131a may be disposed on any one of the substrate upper surface 112a, the substrate lower surface 112b, and the partition wall 112c of the well 111b, or may be disposed on only the substrate upper surface 112a and the substrate lower surface 112b. In this case, photothermal film 131a may be deposited in a pattern.

The photothermal film 131a may receive light from, e.g., a light source of an optical heater of the temperature controller 120, and/or a light source of the optical unit 150, and may generate heat by photonic heating using the received light. In this case, as shown in FIG. 2C, as the photothermal film 131a is disposed at a plurality of positions of the gene amplification chip 110b of the photothermal film 131a, temperature may be controlled at a uniform level, and heat generation efficiency may be improved.

A thickness of the photothermal film 131a may be 10 μm or less, but is not limited thereto. Further, the photothermal film 131a may be formed as a metal layer, but is not limited thereto and may be made of a metal oxide material, metalloid, and base metal. For example, the photothermal film 131a may be formed of a tungsten-based material having excellent infrared absorptivity, and thus achieving a photothermal conversion effect during laser emission. The photothermal film 131a may have a nanostructure. For example, the photothermal film 131a may be formed as nanoparticles, nanorod, nanodisc, or nanoisland, which has a size of 50 nm or less in diameter and 50 nm or less in thickness, but is not limited thereto, and may be formed in various nanostructures. Further, the photothermal film 131a may further contain carbon black, visible light dye, ultraviolet dye, infrared dye, fluorescent dye, radiation-polarizing dye, pigment, metallic compound, and another suitable absorber material as a photothermal conversion material.

The gene amplification chip may further include: an adhesive layer disposed between the substrate 112 and the photothermal film 131a and improving adhesive strength of the photothermal film 131a; an auxiliary film disposed to surround the photothermal film 131a and preventing hindrance to the gene amplification process within the through hole of the photothermal film 131a; and other material for amplifying the photothermal effect of the photothermal film 131a. In this case, the photothermal film 131a, the adhesive layer, the auxiliary film, and the like may be provided by chemical vapor deposition (CVD), Physical Vapor Deposition (PVD), Atomic Layer Deposition (ALD), sputtering, evaporation, and the like, but the method is not limited thereto.

Referring back to FIG. 2A, the electrical heating element 132 may include a Peltier element, but is not limited thereto.

According to elapsed time, the temperature controller 120 may control the temperature of the wells 111a to be each of a thermal dissolution temperature, a reverse transcription temperature, and a gene amplification temperature. For convenience of explanation, the following description will be given using the photothermal film, which is an example of the heat source 130, but is not limited thereto.

The temperature controller 120 may include the wells 111b of the gene amplification chip 110b and/or one or more light sources for emitting light onto the photothermal film disposed in the wells 111b. The light source included in the optical heater may include a light emitting diode (LED), laser, Vertical-cavity surface-emitting laser (VCSEL), and the like, but is not limited thereto. In this case, the optical heater may share the light source of the optical device 150 to emit light onto the wells 111b and/or the photothermal film disposed in the wells 111b.

The temperature controller 120 may adjust heat generated by the photothermal film by controlling the intensity of light included in the optical heater and/or the optical device 150. In this case, the temperature controller 120 may measure a change in temperature of the wells 111b due to heat generated by the photothermal film based on the temperature sensor as shown in FIGS. 1C and 1D, thereby controlling the temperature of wells 111b to be each of the thermal dissolution temperature, the reverse transcription temperature, and the gene amplification temperature, according to elapsed time as shown in FIGS. 1C and 1D.

The time controller 140 may control at least one of a thermal dissolution time, a reverse transcription time, and a gene amplification time of the apparatus 200 for gene amplification by using a timer and the like.

For example, the time controller 140 may control the thermal dissolution time, the reverse transcription time, and the gene amplification time of the apparatus 200 for gene amplification by directly controlling a time of light emission at a specific intensity that is controlled by the temperature controller 120 and emitted by the light source of the optical heater and/or the optical device 150, or by being electrically connected to the time controller 120 and transmitting, to the temperature controller 120, a time of maintaining a specific temperature controlled by the temperature controller 120.

For example, referring to FIG. 1C, if the time controller 140 controls the thermal dissolution reaction during a period from a start point 0 to T1, the temperature controller 120 may control the temperature of the wells 111b to be a thermal dissolution temperature F1 during the period from the start point 0 to T1. Then, if the time controller 140 controls the reverse transcription reaction during a period from T1 to T2, the temperature controller 120 may control the temperature of the wells 111b to be the reverse transcription temperature during the period from T1 to T2.

In this case, the time controller 140 controls the thermal dissolution time to be 10 minutes or less (e.g., one second to 10 minutes), the reverse transcription time 20 minutes or less (e.g., one second to 20 minutes), and the gene amplification time to be 120 minutes or less (e.g., one second to 120 minutes), but the thermal dissolution time, the reverse transcription time, and the gene amplification time are not limited thereto, and may be changed to various values. In this case, if the gene amplification reaction is the PCR, the time controller 140 may control, in each cycle, a time of denaturation to be 15 sec. or 30 sec., a time of annealing to be 30 sec., and a time of extension to be 30 sec., but are not limited thereto, and the time of each process may be changed variously.

While the gene amplification reaction is performed in the wells 111b of the optical device 150, or after the gene amplification is complete, the optical device 150 may measure an optical signal from a sample of the wells 111b. In this case, the optical signal may include fluorescence, phosphor, absorbance, surface plasmon resonance, and the like. The optical device 150 may include a light source for emitting light onto the sample of the wells 111b, and a detector for detecting the optical signal reflected from the sample of the wells 111b.

The light source may include LED, Vertical-cavity surface-emitting laser (VCSEL), and the like, but is not limited thereto. Further, the detector may include a photomultiplier tube, a photo detector, a photomultiplier tube array, a photo detector array, a complementary metal-oxide semiconductor (CMOS) image sensor, and the like, but is not limited thereto.

In this case, the detector may detect fluorescence emitted from an amplified gene by using fluorescence reflected from the photothermal film. For example, when the photothermal film, made of a material having high reflectivity, is disposed on the partition wall of the well 111b having a through hole shape of the gene amplification chip 110b, the photothermal film may reflect fluorescence, emitted from the gene amplified within the through hole, toward the detector. In this case, the detector may detect the fluorescence reflected from the photothermal film.

The optical device 150 may further include a filter for passing light of a specific wavelength, a mirror for adjusting the light emitted from the wells 111b toward the detector, a lens for collecting light emitted from the wells 111b, and the like. The optical device 150 may be omitted, and quantitative analysis of the amplified genes may be performed by an external device.

The processor 160 may be electrically connected to the optical device 150, and may control driving of the light source of the optical device 150. Further, by receiving the optical signal from the detector, the processor 160 may analyze the optical signal. For example, the processor 160 may perform quantitative analysis of an amplification result of genes detected by the detector based on Poisson distribution. At least some of the aforementioned functions of the temperature controller 120 and the time controller 140 may be integrated into the processor 160.

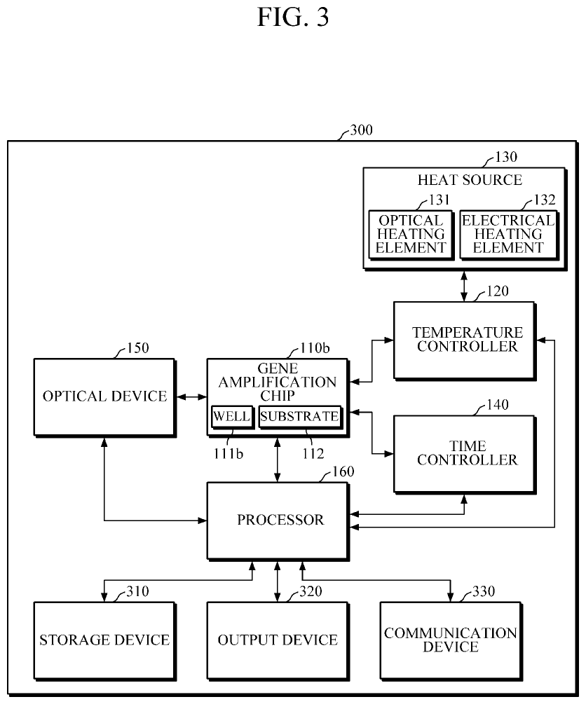

FIG. 3 is a block diagram of an apparatus for gene amplification according to yet another embodiment.

Referring to FIG. 3, the apparatus 300 for gene amplification may further include a storage 310, an output device 320, and a communication device 330, in addition to the components shown in the embodiment of the FIG. 2A.

The storage 310 may store for example, a variety of reference information for gene amplification and/or a gene amplification result, and the like. The storage 310 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

The output device 320 may output, for example, a gene amplification process, gene amplification, an analysis result, and the like. The output device 320 may provide a user with information by visual, audio, and tactile methods and the like using a visual output module (e.g. display), an audio output module (e.g., speaker), a haptic module, and the like.

The communication device 330 may communicate with an external device. For example, the communication device 330 may transmit data generated by the apparatuses 100, 200, and 300 for gene amplification, e.g., a gene detection result, and the like to an external device, and may receive data required for gene detection from the external device. In this case, the external device may be medical equipment, a printer to print out results, or a display device. In addition, the external device may be a digital TV, a desktop computer, a mobile phone, a smartphone, a tablet PC, a laptop computer, Personal Digital Assistants (PDA), Portable Multimedia Player (PMP), a navigation device, an MP3 player, a digital camera, a wearable device, etc., but is not limited thereto.

The communication device 330 may communicate with the external device by using Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, Radio Frequency Identification (RFID) communication, 3G, 4G, and 5G communications, and the like. However, this is merely exemplary and is not intended to be limiting.

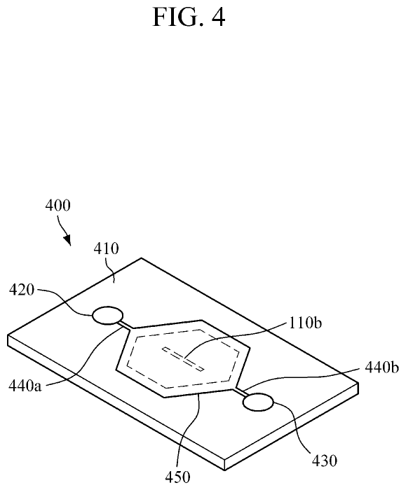

FIG. 4 is a perspective view of an apparatus for gene amplification according to an embodiment.

Referring to FIG. 4, an apparatus 400 for gene amplification may include a main body 410, a sample inlet 420, a sample outlet 430, a chamber 450 disposed on one surface of the main body 410 and connected to the sample inlet 420 and the sample outlet 430 by stream tubes 440a and 440b, and a gene amplification chip 110b inserted into the chamber 450. The main body 410 may have a groove into which the chamber 450 may be inserted.

A sample may be injected through the sample inlet 420. In this case, the sample may include a duplex of one or more of ribonucleic acid (RNA) virus, deoxyribonucleic acid (DNA) virus, peptide nucleic acid (PNA) virus, and locked nucleic acid (LNA) virus, and microbes such as bacteria, pathogen, germ, oligopeptide, protein, toxin, etc., but the sample is not limited thereto.

The sample inlet 420 may include at least one of a field effect transistor (FET), a silicon (Si) photonic structure, a 2D micro/nano material/structure, and the like. Further, the sample inlet 420 may include a structure having optical or electrical heat generation characteristics. For example, the sample inlet 420 may include, for example, an optical heating material/structure, which reacts to a light source such as LED, laser, VCSEL, and the like, or an electrical heating element such as the Peltier element, and the like.

FIG. 4 shows the sample inlet 420 having a circular shape, but the size, shape, and amount of the sample inlet 420 are not limited thereto, and may be changed variously. Further, FIG. 4 shows an arrangement in which the sample inlet 420 and the sample outlet 420 are disposed horizontal and parallel to the chamber 450, but the arrangement is not limited thereto, and may be changed variously, as in the case where the sample inlet 420, the chamber 450, and the sample outlet 430 may be vertically disposed, and the like.

The apparatus 400 for gene amplification may further include a storage containing reactants for each gene to be amplified. In this case, the reactants for each gene may include reverse transcriptase, polymerase, ligase, peroxidase, primer, probe, etc., but is not limited thereto. The primer may include oligonucleotide, for example, target specific single strand oligonucleotide. Further, the probe may include oligonucleotide, for example, target specific single strand oligonucleotide, a fluorescent material, quencher, and the like. The probe may exhibit a fluorescence signal by interacting with a specific target material in a solution, in which different types of materials are dissolved. Such characteristic signal may be tracked, detected, and processed for a predetermined period of time by a detector and/or a processor of the apparatus for gene amplification, to be used in gene detection.

The sample injected by the apparatus 400 for gene amplification may flow into the chamber 450 along the stream tube 440a. In this case, the stream tubes 440a and 440b may include a valve for controlling a flow of the sample. In this case, the valve may be various types of microvalve for opening and closing the stream tubes 440a and 440b. For example, the microvalve may be an active microvalve, such as a pneumatic/thermopneumatic actuated valve, an electrostatically actuated valve, a piezoelectrically actuated valve, an electromagnetically actuated valve, etc., or a passive microvalve for opening and closing the tubes based on a direction of a fluid flow or an interfacial tension difference, without artificial external operation, but the microvalve is not particularly limited.

The stream tube 440a may further include a filter for blocking fine particles in the sample loaded into the sample inlet 420 and preprocessed, and for passing only a fluid. The filter may be formed in a single layer or multilayer film shape having microholes, and may block the fine particles of a desired size according to the size of the holes. The filter may be made of, for example, silicon, polyvinylidene fluoride (PVDF), polyethersulfone, polycarbonate, glass fiber, Polypropylene, Cellulose, Mixed cellulose esters, Polytetrafluoroethylene (PTFE), Polyethylene Terephthalate, Polyvinyl chloride (PVC), Nylon, Phosphocellulose, Diethylaminoethyl cellulose (DEAE), and the like, but is not limited thereto. The hole may have various shapes, e.g., a circular shape, a rectangular shape, a slit shape, an irregular shape due to glass fiber, and the like.

In FIG. 4, the stream tubes 440a and 440b are disposed on left and right sides, respectively, of the chamber 450 in a straight line, but are not limited thereto. For example, the stream tubes 440a and 440b may be disposed in various curved shapes, and may include a plurality of channels.

The aforementioned sample inlet 420 and stream tubes 440a and 440b may be omitted, in which case the sample may be directly loaded into the gene amplification chip 110b in the chamber 450 without the sample inlet and the stream tubes.

The injected sample may flow into the chamber 450 along the stream tube 440a by capillary action. However, unlike the example, the apparatus 400 for gene amplification may further include an active/passive driving device, a structure for moving the sample such as an electro-wetting device, and the like. In this case, the active/passive driving device may include a passive vacuum void pump, a syringe pump, a vacuum pump, a pneumatic pump, etc., but is not limited thereto.

The chamber 450 may include an upper surface (not shown) and a lower surface, and the gene amplification chip 110b may be inserted between the upper surface and the lower surface of the chamber 450. In this case, the upper surface and the lower surface of the chamber 450 may be glass layers, but are not limited thereto, and may be formed of various materials.

The light source of the optical device of the apparatus 400 for gene amplification may emit light onto the sample of the gene amplification chip 110b inserted into the chamber 450, and the detector 450 may detect an optical signal reflected from the sample of the gene amplification chip 110b inserted into the chamber 450.

A sample, introduced into the chamber 450 but not loaded into the gene amplification chip 110b, or a sample, for which the gene amplification process has been completed, may be discharged to the sample outlet 430 along the stream tube 440b.

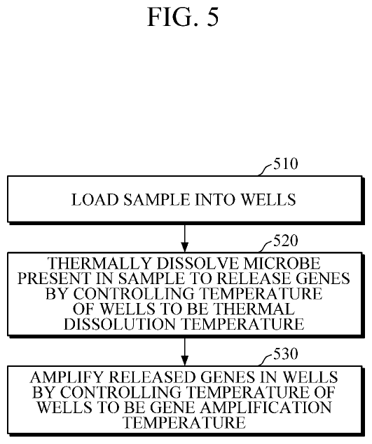

FIG. 5 is a flowchart of a method of gene amplification according to an embodiment.

The method of gene amplification of FIG. 5 may be performed by the apparatuses 100, 200, and 300 for gene amplification according the embodiments of FIGS. 1A, 2, and 3, which are described above in detail, and thus will be briefly described below in order to avoid redundancy.

The apparatus for gene amplification may load a sample into wells in operation 510.

By controlling temperature of the wells to be a thermal dissolution temperature, the apparatus for gene amplification may thermally dissolve a microbe present in the sample to release genes in operation 520. In this case, the microbe may be DNA virus, RNA virus, or bacteria, but is not limited thereto. A viral membrane or shell of the microbe may be removed by the thermal dissolution. For example, if the microbe is the DNA virus, the viral membrane of the DNA virus may be removed by the thermal dissolution, such that DNA, i.e., genes inside the virus, may be released. The thermal dissolution temperature may be predetermined according to the type of microbe to be loaded, or may be changed to various values according to a user's manipulation. The thermal dissolution temperature may be an isothermal temperature during a thermal dissolution time, but is not limited thereto, and may increase or decrease within a predetermined range. In this case, the thermal dissolution time may be controlled to 10 minutes or less (e.g., e.g., one second to 10 minutes).

Subsequently, by controlling the temperature of the wells to be a gene amplification temperature, the released genes may be amplified in the wells in operation 530. For example, if the microbe is the DNA virus, DNA released during the thermal dissolution may be amplified in the wells.

The gene amplification reaction may include at least one of polymerase chain reaction (PCR) amplification and isothermal amplification, but is not limited thereto. In this case, according to the type of gene amplification reaction, the temperature controller 120 may vary the gene amplification temperature of the wells 111a. For example, if the gene amplification reaction is the PCR amplification, the temperature controller 120 may control the temperature of the wells 111a based on thermal cycling, and if the gene amplification reaction is the isothermal amplification, the temperature controller 120 may control the temperature of the wells 111a to be maintained at a specific temperature. In this case, the gene amplification time may be controlled to 120 minutes or less (e.g., one second to 120 minutes). In this case, while the gene amplification reaction is performed in the wells, or after the gene amplification is complete, the apparatus for gene amplification may measure an optical signal from a sample of the wells, and may perform quantitative analysis of the amplified gens by using the measured optical signal. In this case, by using the light source of the optical device for emitting light of a predetermined wavelength onto the wells, and the apparatus for gene amplification may detect an optical signal, such as fluorescence, phosphorescence, absorbance, surface plasmon resonance, etc., radiating from the sample of the wells, and may perform quantitative analysis of the amplified genes based on the detected optical signal and Poisson distribution. A detailed description thereof will be omitted.

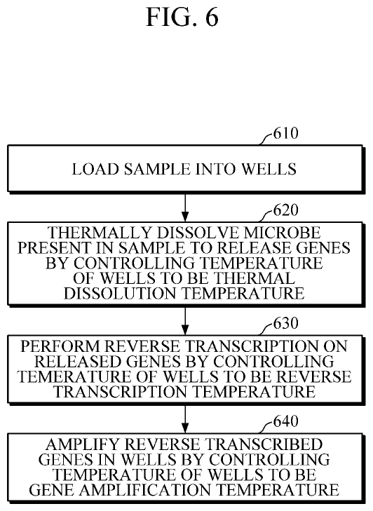

FIG. 6 is a flowchart of a method of gene amplification according to another embodiment.

The method of gene amplification of FIG. 6 may be performed by the apparatuses 100, 200, and 300 for gene amplification according the embodiments of FIGS. 1A, 2, and 3, which are described above in detail, and thus will be briefly described below in order to avoid redundancy.

The apparatus for gene amplification may load a sample into wells in operation 610.

By controlling temperature of the wells to be a thermal dissolution temperature, the apparatus for gene amplification may thermally dissolve a microbe present in the sample to release genes in operation 620. In this case, the microbe may be RNA virus. In this case, the viral membrane of the RNA virus may be removed by the thermal dissolution, such that RNA, i.e., genes inside the virus, may be released.

For example, the apparatus for gene amplification may control the thermal dissolution temperature based on the reverse transcription temperature. For example, the apparatus for gene amplification may control the thermal dissolution temperature based on a predetermined activation temperature range of a reverse transcriptase used to perform reverse transcription. The thermal dissolution temperature may be an isothermal temperature within the predetermined activation temperature range of the reverse transcriptase, or may increase or decrease within a predetermined range.

In this case, if the thermal dissolution temperature required for a microbe, e.g., RNA virus, exceeds the predetermined activation temperature range of the reverse transcriptase, a viral membrane softening agent may be used. The viral membrane softening agent may include ethanol, isopropyl alcohol, methanol, 1-propanol, 2-propanol, 1-butanol, 2-butanol, triethylamine, dimethylformamide, hexamethylphosphoric triamide, dimethyl sulfoxide, acetone, acetonitrile, pyridine, metal particle, and organic solvent such as detergent, ethyl acetate, hexanol, and the like, but is not limited thereto.

By controlling the temperature of the wells to the reverse transcription temperature, the apparatus for gene amplification may perform reverse transcription on the released genes in operation 630. For example, if the microbe is the RNA virus, the apparatus for gene amplification may perform reverse transcription on the RNA, released during the thermal dissolution, by using the reverse transcriptase. The reverse transcription temperature may be an isothermal temperature within the predetermined activation temperature range of the reverse transcriptase, or may increase or decrease within a predetermined range. The reverse transcription time may be controller to 20 minutes or less (e.g., one second to 20 minutes). A detailed description thereof will be omitted.

By controlling the temperature of the wells to the gene amplification temperature, the apparatus for gene amplification may amplify the reverse transcribed genes in the wells in operation 640. The gene amplification reaction may include at least one of polymerase chain reaction (PCR) amplification and isothermal amplification, but is not limited thereto, and according to the type of gene amplification reaction, the apparatus for gene amplification may vary the gene amplification temperature of the wells. A detailed description thereof will be omitted.

The present disclosure can be realized as a computer-readable code written on a computer-readable recording medium. The computer-readable recording medium may be any type of recording device in which data is stored in a computer-readable manner.

Examples of the computer-readable recording medium include a ROM, a RAM, a CD-ROM, a magnetic tape, a floppy disc, an optical data storage, and a carrier wave (e.g., data transmission through the Internet). The computer-readable recording medium can be distributed over a plurality of computer systems connected to a network so that a computer-readable code is written thereto and executed therefrom in a decentralized manner. Functional programs, codes, and code segments needed for realizing example embodiments can be easily deduced by computer programmers of ordinary skill in the art.

The present disclosure has been described herein with regard to example embodiments. However, it will be obvious to those skilled in the art that various changes and modifications can be made without changing technical ideas and essential features of the present disclosure. Thus, it is clear that the above-described embodiments are illustrative in all aspects and are not intended to limit the present disclosure.

What is claimed is:

1. An apparatus for gene amplification, the apparatus comprising:
a gene amplification chip comprising a substrate including a well the well configured to accept a sample that is loaded into the well; the gene amplification chip being configured to:
thermally dissolve a microbe present in the sample, to release genes in the microbe, by heating the sample in the well to a thermal dissolution temperature; and
amplify the released genes by heating the sample in the well to a gene amplification temperature; and
a temperature controller configured to control a temperature of the well to be the thermal dissolution temperature and the gene amplification temperature.

2. The apparatus of claim 1, wherein a diameter of the well is larger than or equal to a diameter of the microbe, and is less than or equal to 10000 times the diameter of the microbe.

3. The apparatus of claim 2, wherein each of the diameter and a depth of the well is in a range of 1 nm to 1000 μm.

4. The apparatus of claim 1, wherein a volume of the well is 1 nL or less.

5. The apparatus of claim 1, further comprising:
an optical device configured to, while the gene amplification is being performed in the well or after the gene amplification is complete, measure an optical signal that is scattered or reflected from the sample in the well; and
a processor configured to detect the amplified genes by analyzing the measured optical signal.

6. The apparatus of claim 1, further comprising a time controller configured to control a time of the thermal dissolution to be 10 minutes or less, and a time of the gene amplification to be 120 minutes or less.

7. The apparatus of claim 1, wherein the gene amplification chip is further configured to, in response to the microbe being RNA virus, reverse transcribe RNA, that is released after the RNA virus is thermally dissolved in the well, using a reverse transcriptase in the well.

8. The apparatus of claim 7, wherein:
the temperature controller is further configured to control the thermal dissolution temperature of the well based on a predetermined activation temperature range of the used reverse transcriptase; and
the apparatus for the gene amplification further comprises a time controller configured to control a time of the reverse transcription to be 20 minutes or less.

9. The apparatus of claim 1, wherein a viral membrane softening agent is used for the thermal dissolution.

10. The apparatus of claim 9, wherein the viral membrane softening agent comprises at least one of ethanol, isopropyl alcohol, methanol, 1-propanol, 2-propanol, 1-butanol, 2-butanol, triethylamine, dimethylformamide, hexamethylphosphoric triamide, dimethyl sulfoxide, acetone, acetonitrile, pyridine, metal particle, detergent, ethyl acetate, or hexanol.

11. The apparatus of claim 1, further comprising at least one heat source of an optical heating element including a photothermal film for generating heat by using received light, and an electrical heating element including a Peltier element,
wherein the temperature controller controls the thermal dissolution temperature and the gene amplification temperature by using the heat source.

12. The apparatus of claim 1, wherein the gene amplification comprises at least one of polymerase chain reaction (PCR) amplification and isothermal amplification.

13. The apparatus of claim 1, wherein the substrate includes in a range of one to a hundred thousand wells, each well being configured to accept, thermally dissolve, and amplify a sample respectively introduced into each well.

14. The apparatus of claim 1, wherein the well has a through hole shape and passes through the substrate in a direction from an upper surface of the substrate to a lower surface of the substrate.

15. A method of gene amplification, the method comprising:
loading a sample comprising a microbe into a well of a substrate;
thermally dissolving the microbe in the loaded sample to release genes from the microbe by controlling a temperature of the well to be a thermal dissolution temperature; and
amplifying the released genes in the well by controlling the temperature of the well to be a gene amplification temperature.

16. The method of claim 15, further comprising:
emitting light onto the sample of the well while the gene amplification is performed in the well or after the gene amplification is complete;
measuring an optical signal that is scattered or reflected from the sample in the well; and
detecting the amplified genes by analyzing the measured optical signal.

17. The method of claim 15, wherein:
the thermally dissolving of the microbe in the well comprises controlling a time of the thermal dissolution to be 10 minutes or less; and
the amplifying of the released genes in the well comprises controlling a time of the gene amplification to be 120 minutes or less.

18. The method of claim 15, further comprising, in response to the microbe being RNA virus, performing reverse transcription on RNA, released after the RNA virus is thermally dissolved in the well, using a reverse transcriptase in the well,
wherein:
the thermally dissolving of the microbe in the well comprises controlling the thermal dissolution temperature of the well based on a predetermined activation temperature range of the used reverse transcriptase; and
the reverse transcription comprises controlling a time of the reverse transcription to be 20 minutes or less.

19. The method of claim 15, wherein the thermally dissolving of the microbe in the well comprises using a viral membrane softening agent.

20. The method of claim 15, wherein the thermally dissolving of the microbe in the well and the amplifying of the released genes in the well comprise controlling the temperature of the well to be the thermal dissolution temperature and the gene amplification temperature, respectively, by using a heat source comprising at least one of:
an optical heating element configured to emit light on a photothermal film on the substrate, the photothermal film being configured to heat the sample in the well by generating heat from the light emitted from the optical heating element; or
an electrical heating element comprising a Peltier element.
